(19) United States
(12) Patent Application Publication
(10) Pub. No.: US 2008/0208629 A1
(43) Pub. Date: Aug. 28, 2008

(54) Medical Information and Data Management System

(76) Inventor: B. Susan Davison, Phoenix, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 11/678,431

(22) Filed: Feb. 23, 2007

(51) Int. Cl. A61B 5/00

(52) U.S. Cl. 705/3

(57) ABSTRACT

A computer-implemented method administers medical care for a plurality of onsite clinics. Preexisting medical data is aggregated to generate a plurality of gross health care expenses which are organized according to each of a plurality of health conditions. Each of the plurality of gross health care expenses is prioritized according to cost to generate a health risk assessment. A pool of associated patients is selected from each of the plurality of gross health care expenses according to a targeted aggregate health care expense. A first patient of the pool of associated patients is identified to determine an appropriate treatment action plan for the first patient. The treatment action plan is consistent with the health risk assessment. Onsite consultation, medical testing and benchmarking are scheduled for the first patient according to the treatment action plan.

### FIELD OF THE INVENTION

The present invention relates in general to computers and, more particularly, to a computer-implemented system and method of improving the administration of medical benefits and enhancing quality of care in patients.

### BACKGROUND OF THE INVENTION

Employers generally provide for, or at least contribute to, medical insurance, and thereby, medical care for nonexempt employees. A typical employer will expend large resources to provide health services to employees. Employers realize that the availability and prudent administration of health benefits to employees is an important expectation of the employer/employee relationship. Most employers also recognize the connection between healthy employees and a productive workforce. As a result, employers pay health related expenses as a cost of doing business.

Health costs, and partly as a result, health care premiums have greatly increased over the years. Employers, saddled with the rising cost of health care, have explored options to reduce costs, while still upholding traditional health care quality that many employees have come to expect. Some recent example changes reflecting the attempt to reduce cost have included the rise in large health maintenance organizations (HMOs) and the advent of flexible health savings accounts.

Even with the changes that have occurred in health benefit administration to combat rising costs, employers and employees have continued to see rising cost, as well as diminishing quality of care. Moreover, employers and many employees have become more interested in preventative healthcare as a viable cost reduction option, an approach which is beginning to be recognized as important, but as of yet, under-implemented.

As a result, a need exists for a cost-effective system and method of administering health benefits, and thereby, health care which manages data effectively and efficiently. Additionally, the system and method should take recent preventative steps into account.

### SUMMARY OF THE INVENTION

In one embodiment, the present invention is a computer-implemented method for administering medical care for a plurality of onsite clinics, comprising aggregating preexisting medical data to generate a plurality of gross health care expenses which are organized according to each of a plurality of health conditions, prioritizing each of the plurality of gross health care expenses according to cost to generate a health risk assessment, selecting a pool of associated patients from each of the plurality of gross health care expenses according to a targeted aggregate health care expense, identifying a first patient of the pool of associated patients to determine an appropriate treatment action plan for the first patient, the treatment action plan consistent with the health risk assessment, and scheduling onsite consultation, medical testing and benchmarking for the first patient according to the treatment action plan.

In another embodiment, the present invention is a computer-implemented method for administering medical care within an onsite clinic, comprising combining insurance claims or self assessment data to generate a plurality of aggregate medical costs organized according to each of a plurality of medical conditions, generating a health risk assessment based on a prioritization of each of the plurality of aggregate medical costs, targeting a patient pool associated with a first aggregate medical cost of the plurality of aggregate medical costs based on a proportion of selected medical expenses of the patient pool in relation to the first aggregate medical cost, selecting a first patient of the patient pool to implement a management plan consistent with the health risk assessment, and implementing a consultation, medical testing, and benchmarking schedule according to the management plan.

In another embodiment, the present invention is an article of manufacture including computer readable program code for administering medical care within an onsite clinic, wherein the code is capable of causing operations to be performed comprising combining insurance claims or self assessment data to generate a plurality of aggregate medical costs organized according to each of a plurality of medical conditions, generating a health risk assessment based on a prioritization of each of the plurality of aggregate medical costs, targeting a patient pool associated with a first aggregate medical cost of the plurality of aggregate medical costs based on a proportion of selected medical expenses of the patient pool in relation to the first aggregate medical cost, selecting a first patient of the patient pool to implement a management plan consistent with the health risk assessment, and implementing a consultation, medical testing, and benchmarking schedule according to the management plan.

In still another embodiment, the present invention is a system for administering medical care for an onsite clinic, comprising a database module for combining preexisting medical data to generate a plurality of aggregate medical costs organized according to each of a plurality of medical conditions, a management module connected to the database module, wherein the management module generates a health risk assessment based on a prioritization of each of the plurality of aggregate medical costs, targets a patient pool associated with a first aggregate medical cost of the plurality of aggregate medical costs based on a proportion of selected medical expenses of the patient pool in relation to the first aggregate medical cost, and selects a first patient of the patient pool to implement a management plan consistent with the health risk assessment, and a scheduling module connected to the management module for implementing a consultation, medical testing, and benchmarking schedule according to the management plan.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates an example computer system on which aspects of the present invention can be implemented;

FIG. 2 illustrates a system according to the present invention, operable on a computer system, for implementing various aspects of the present invention;

FIG. 3 is a conceptual illustration of an example chronic illness and care flowchart in which aspects of the present invention can be implemented;

FIG. 4 illustrates a first example method of operation of the medical information and data management system of FIG. 2; and

FIG. 5 illustrates a second example method of operation of the medical information and data management system of FIG. 2.

### DETAILED DESCRIPTION OF THE DRAWINGS

Some of the functional units described in this specification have been labeled as modules in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like.

Modules may also be implemented in software for execution by various types of processors. An identified module of executable code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Reference to a signal bearing medium may take any form capable of generating a signal, causing a signal to be generated, or causing execution of a program of machine-readable instructions on a digital processing apparatus. A signal bearing medium may be embodied by a transmission line, a compact disk, digital-video disk, a magnetic tape, a Bernoulli drive, a magnetic disk, punch card, flash memory, integrated circuits, or other digital processing apparatus memory device.

The schematic flow chart diagrams included are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Implementation of various aspects of the present invention provides a tool for employers, businesses and other organizations to provide better quality of medical care, including care having a preventative focus instead of a reactionary focus. In the process, employers can significantly reduce medical costs, both to the employer and to the employee.

In one embodiment, aspects of the present invention can be implemented through the use of an “onsite” medical clinic. Reference to “onsite clinics” can include various facilities having a mission to provide medical care to patients. The facilities are located at an onsite employer location, as opposed to being located at a location removed from the place of employment.

In addition to using onsite clinics, the present invention can coordinate the services of various medical professionals. One embodiment of the present invention, for example, contemplates the use of “nurse practitioners.” A nurse practitioner (NP) is generally a registered nurse who has completed advanced education (generally a minimum of a master's degree) and training in the diagnosis and management of common medical conditions, including chronic illnesses. Nurse practitioners can provide a broad range of health care services as will be described below.

Nurse practitioners can provide much of the same care provided by physicians and usually maintain close working relationships with physicians. An NP can serve as a patient's regular health care provider and see patients of all ages. Nurse practitioners are in a unique position to provide individualized care. Nurse practitioners can focus on patients' conditions as well as the effects of illness on the lives of the patients and families of the patient. NPs can help make prevention, wellness, and patient education priorities. The use of NPs in many cases can mean fewer prescriptions and less expensive treatments.

NPs can inform patients about health care and encouraging the patient to participate in decisions relating to healthcare. In addition to health care services, NPs can conduct research and are often active in patient advocacy activities.

NPs can work autonomously and some are able to open their own clinical practices. American NPs, according to the American College of Nurse Practitioners, are required in 28 states to practice in collaboration with a physician though they may prescribe medications and carry a DEA number in 49 states. Because the profession is state regulated, care provided by NPs varies.

A nurse practitioner's duties can include diagnosing, treating, evaluating and managing acute and chronic illness and disease (e.g., pneumonia, diabetes, high blood pressure), obtaining medical histories and conducting physical examinations, ordering, performing, and interpreting diagnostic studies (e.g., lab tests, x-rays, EKGs), prescribing medications, prescribing physical therapy and other rehabilitation treatments, providing prenatal care and family planning services, providing well-child care, including screening and immunizations, providing primary and specialty care services, health maintenance care for adults, including annual physicals, performing minor surgeries and procedures (e.g., dermatological biopsies, suturing, casting), collaborating with physicians and other health professionals as needed, including providing referrals, and finally, counseling and educating patients on health behaviors, self-care skills, and treatment options.

Medical professionals such as NPs can be positioned in an onsite environment to provide a high level of personalized and diversified care at lower overall cost. Through the use of onsite clinics, a variety of services can be provided to employees. The services normally would have to be provided through a typical medical office. A system can be implemented to manage an onsite environment, where a medical professional can be scheduled to become responsible for a certain population of employees.

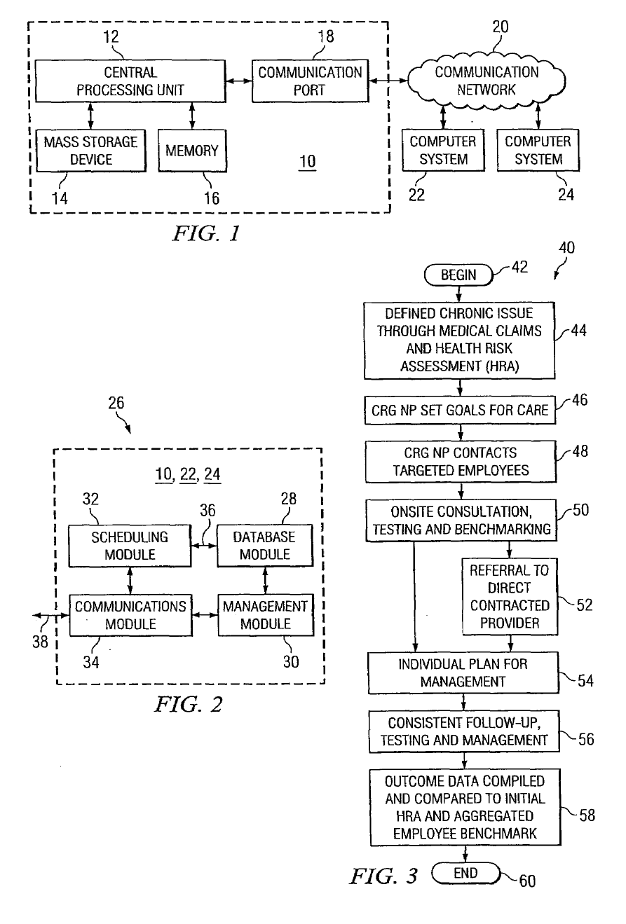

Turning to FIG. 1, an example computer system for implementing various aspects of the present invention is depicted. System 10 includes a central processing unit (CPU) 12, which is connected by a signal bearing medium to a mass storage device 14 such as a hard disk drive or similar nonvolatile memory. CPU 12 is also connected to memory 16, which can include such memory devices as flash memory. A communication port 18 connects CPU 12 to a communication network 20, which is depicted external to system 10 in a wide-area-network (WAN) configuration, although the network 20 can include such internal network topologies as local area networks (LANs). Finally, computer system 10 is depicted linked to remote computer systems 22, 24 via communication network 20. The methods described herein can execute on one or more computer systems 10, 22, 24, or elsewhere using hardware described, software or firmware compatible with such hardware, or a combination of hardware, software and firmware components.

For example, the methods described herein can execute using such commonly used software platforms as Windows® or Linux. The methods can be implemented with such software applications as Java®. The methods can be embodied in a computer program product including a computer readable medium having programmable instructions executable on systems 10, 22, 24 or elsewhere. The computer program product can be delivered to systems 10 via remote access, over communication network 20 in mediums such as the world-wide-web (WWW), or from remote servers 22 to local workstations 10 over a proprietary or private network 20. Updates to system 10 can be received over network 20.

In addition, system 10 can include portable, handheld devices such as cellular phones, laptop computers, personal desktop assistant devices (PDAs), Blackberry® devices, and the like. The signal bearing medium connecting system 10 with the communication network can be wireless in nature, such as an 802.11(a)-(e) wireless communications protocol or similar. As a result, a health care practitioner such as a NP can receive the flexibility to utilize various aspects in a variety of locations and situations.

FIG. 2 illustrates various example subcomponents which can be organized according to the present invention to perform medical data and patient management functions. The subcomponents 26 can be operational on a host of computer systems such as those depicted in systems 10, 22, or 24. The various functions of the subcomponents 26 will be described.

A database module 28 provides storage and retrieval of medical information, Database module 28 contains an organized collection of data. Module 28 can be organized according to a variety of models, such as a flat, hierarchical, network, or relational. Module 28 can incorporate such standards as structured query language (SQL) or similar standards known in the art. The various database platforms which can constitute module 28 can utilize software database drivers having application programming interfaces (APIs), examples of which include Java database connectivity (JDBC). The APIs can function to retrieve information stored in module 28 or any of the various subcomponents of module 28.

Module 28 can also have various subcomponent databases. Module 28 can include additional databases or utilize organizational structures as needed to satisfy a particular application, such as a relational database subcomponent to organize identification information associated with a particular patient. The module 28 can include information about patients, medical information, drug schedules, medical expenses, claims histories, and patient survey information, as well as information such as medical expense histories, appointment histories, case histories, drug indications, and a host of other information. As will be seen, a system 10 can utilize the medical information and related data to perform various operations according to the present invention.

Database module 28 is shown connected to management module 30 through one of several signal bearing mediums 36. Management module 30 can work closely with database module 28 to coordinate the use of medical information. In one embodiment, management module 30 can generate a health risk assessment based on a prioritization of each of a plurality of aggregate medical costs. Module 30 can target a patient pool based on a variety of factors which will be further described. Additionally, module 30 can select first patient of the patient pool to implement a management plan consistent with the health risk assessment.

Database module is shown connected to scheduling module 32 through medium 36. Scheduling module 32 works with database module 28 and management module 30 to implement various scheduling functions. For example, module 32 can schedule an initial consultation, or implement a drug delivery schedule. Module 32 can implement a follow-up schedule or initiate a benchmarking activity conducted through system 10.

Finally, communications module 34 is shown connected to scheduling module 32 and management module 30. Communications module 34 can implement communications-related functionality, such as coordinating the display of various scheduling activities on a graphical user interface (GUI) which is interpreted by a NP to implement a treatment plan for a particular patient. Communications module 34 can remind a medical professional to conduct an assessment activity, for example. Communications module 34 can communicate through medium 38 to an external system 10 or other processing component. Module 34 can implement an appointment message which is communicated to an employee via the employee's workstation. The employee can, in turn, respond to the appointment message, accept the message, and send a response through medium 38 to module 34 where an appointment is scheduled by the scheduling module 32.

Turning to FIG. 3, a conceptual illustration of an example chronic illness and care flowchart 40 in which aspects of the present invention can be implemented is depicted. Flowchart 40 begins (step 42) by the step of identifying and defining a chronic health issue through the use of medical claims and the generation of a health risk assessment. Medical information, such as insurance claims information, can be aggregated in database module 28. Statistical information, such as a table of medical expenses, can be generated in spreadsheet form. The medical expenses can be organized according to a predefined list of medical conditions. The medical conditions can correlate to a diagnosis code which appears as part of a patient's medical information, for example. The medical expenses can be organized for the convenience of the employer or for an administrator.

A health risk assessment (HRA) can be generated based upon the statistical information which is gathered. The HRA can identify certain groups of medical conditions which are consuming an inordinate amount of medical expense dollars, for example. The HRA can prioritize medical conditions based on cost or projected costs of treatment, for example.

In one embodiment, system 10 correlates with a medical professional such as an NP to set and implement goals (step 46) for patient care. An NP can be given responsibility for a certain population of patients within an onsite location. The system 10 can implement statistical algorithms to identify individual patients within the population to target for specialized medical care. The targeted employees are contacted by the system 10 (step 48). System 10 schedules and implements a series of onsite activities, including initial and follow up consultations and related clinic appointments, appropriate medical testing (including advanced testing where necessary), and initial and follow up benchmarking activities (step 50).

In some cases, a targeted employee patient will need to be referred to a participating medical provider for advanced treatment. For example, a blood test may reveal the need for further screening which can only be performed at a specialized medical site. In these cases, system 10 can refer the patient to a direct contracted medical provider (step 52).

In any case, a series of individual medical treatment plans can be developed by system 10 which are tailored by the system 10 to be appropriate for each respective patient (step 54). In addition, the treatment plans can be managed by the system to include a variety of preventative measures of treatment. For example, a patient with high cholesterol can be provided not only with a cholesterol reducing drug regimen for a period of time, but with specialized instruction by a medical professional such as a NP as to how the patient can reduce risk.

For successful implementation of system 10 to a patient population or patient pool within an onsite location, the need for consistent follow up, additional testing where appropriate, and overall case management is a continuing concern (step 56). System 10 assists NPs and other medical professionals to implement a medical treatment plan which is individualized for each employee patient, and yet is consistent with an overall HRA set for the onsite location.

System 10 can perform adaptive tasks in order to provide consistent and effective health care management. The system 10 can generate and analyze outcome data which is compiled and compared to the initial HRA (step 58). In addition, the system 10 can aggregate employee benchmarking data which can also be compared with the HRA. By comparing medical information on an ongoing basis, the system 10 can adapt in scheduling and implementing health care to provide greater effectiveness in treatment, particularly on a long-term basis. Flowchart 40 then ends (step 60).

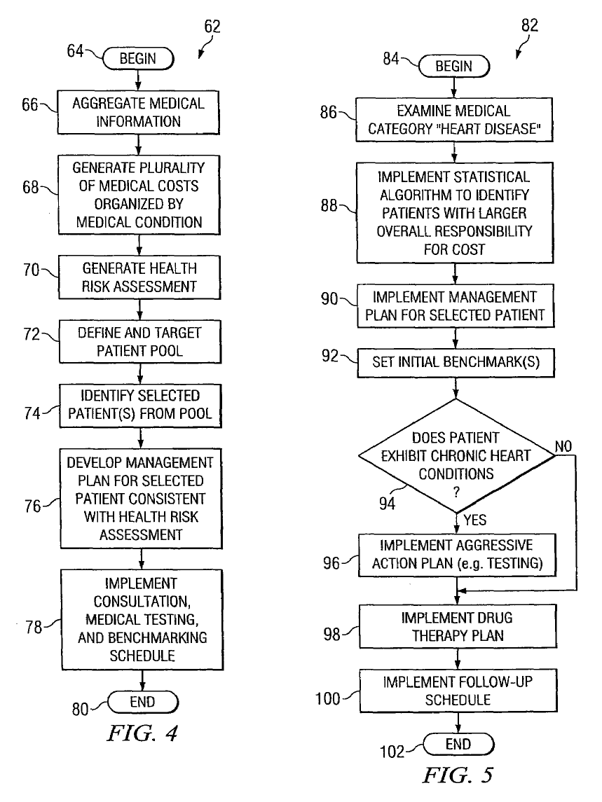

FIG. 4 illustrates an example computer-implemented method 62 of operation consistent with the present invention. Method 62 begins (step 64) by the aggregation of medical information as previously described (step 66). In one embodiment, a plurality of medical costs are generated which can be organized by medical condition, depending on the particular situation (step 68).

As a next step, a health risk assessment (HRA) is generated (step 70). A patient pool or patient population is defined and targeted (step 72). Selected patient(s) are identified and selected for a particular course of treatment (step 74). A management plan for the respective selected patient is developed which is consistent with the initial HRA (step 76). A consultation, medical testing, and benchmarking schedule is generated and implemented (step 78). Method 62 then ends (step 80).

With the addition of an onsite healthcare facility, employers can have control of cost of care traditionally delivered. The reduction of health care cost again can extend to chronic, preventable (manageable) conditions. Consider the following example A of how system 10 can implement various aspects of the present invention to more effectively manage health care in an onsite environment.

In assessing the current cost of chronic, preventable conditions, system 10 can again use both aggregated data as well as individual (both in claims and self assessment) data to determine the current hard dollar cost of the conditions. A sample company (for the present example hereinafter “Employercorp”) has various medical data which is gathered and aggregated. System 10 determines aggregated costs of $600,000 per year of Employercorp in: Heart Disease, Diabetes, Weight related disease, Asthma, Vascular disease, and Cerebrovascular disease. Of these chronic conditions, system 10 determines that Heart Disease makes up 63% or the total or $378,000 per year.

In addition to the foregoing aggregate medical expenses, individual assessments are used to specifically target those incurring the cost or have the potential to incur higher future costs. System 10 implements statistical methods and algorithms to, in effect, “drill down” on the Heart disease costs and determine 28 individuals who are responsible for 88% of the cost to treat their heart related conditions.

System 10 generates an initial treatment action plan which will utilize actual claim data and health risk assessments (HRA) to determine which employees to contact and the management plan designed for each individual. In most cases, the direction of care will be setting initial benchmarks and managing care through drug therapy and consistent follow-up which is scheduled through system 10. Again, consistent follow-up is important to implement as the onsite Nurse Practitioner (NP) and employee will have daily access to each other with the NP proactively setting consistent follow up testing dates. In more extreme chronic heart related conditions the NP will determine a more aggressive action plan, which will include referrals to additional high level testing.

Both insured and self-insured health plans offered through employers provide extensive care, however, the provider network utilized by the Employercorp's plan can limit the level of care in both physician and facility access. Standard levels of care are always available, yet there are a significant number of excluded diagnostic procedures, not to mention a limit on the number of providers and testing facilities.

Health insurers and self-funded employers also contractually exclude or extensively limit specific benefits, which are considered experimental, too costly (when less costly standard care is available), or not medically necessary.

Returning to example A, Employercorp's self-funded exclusions and limitations extend to high-level heart testing and heart scans. To offset costs and make available the highest level of care, an administrator of system 10 works directly with medical providers and facilities to negotiate Direct Contracts. The contracts are between the employer and the facility providing the testing and care. For example, Local Heart Institute is a very high level provider in physician care, state of the art testing and management of heart related issues. The institute is a contracted provider for standard care; however, the advanced, high level testing (e.g., 64 Slice Heart Scan Imaging) is excluded. The administrator contracts with the facility to perform the 64 slice procedure for Employercorp's members for a fraction of the retail cost. Additionally, required procedures based on testing results take place within the institute, the highest level of care available.

As a result of the implementation of the direct contracts, a medical professional such as an NP has access to advanced testing to which she can refer high-level, chronic patients. Coordination of system 10 with a medical professional such as an NP implements a gatekeeper function to access preferred pricing and hold overall costs down.

Traditional employer health plans offer one or more plans in which the members make personal decisions on how/when to access care. This is predominantly the case after a health issue appears. If the health issue is the beginning of a potential chronic illness, the patient makes the buying decision on how that care is accessed, if at all. The potential for the illness to become a high claim incident is likely.

Instead, the present invention implements a scheme which includes, first, intervention of existing known chronic health issues, management of care through consistent onsite interaction, and advancement of care management when appropriate. More importantly, system 10 can work to proactively evaluate the worksite population to catch individuals who have the potential for chronic illness and begin the management long before the illness would have otherwise appeared.

In terms of dollars, the onsite clinic will function as the education, testing, and health coach facility for all employees. This allows employees to stay at work, have easier/consistent access to care, manage both health and illness more efficiently and have access to higher levels of care when needed. Long-term savings will be seen in fewer high dollar chronic illness claims and ultimately medical claims that grow at a much slower pace than traditional employer health plans.

Turning to FIG. 5, an example method 82 of operation, according to example A, is depicted. Method 82 begins (step 84) by a close examination of the Heart Disease health category (step 86) which is identified as a condition having a significant contribution to the overall health cost of Employercorp. Heart Disease is identified having a significant percentage of the conditions defined as “preventable.” As a result, Heart Disease can be organized as a condition having a higher priority to Employercorp's HRA. Other preventable chronic conditions include asthma, diabetes, weight-related disease, vascular disease and cerebrovascular disease. Chronic conditions such as the foregoing can be predefined as preventable, or organized in a similar way. In addition to examining preventable conditions, however, an embodiment of method 82 can examine all health conditions, or a selected body of health conditions taken from a predetermined body of health conditions which may or may not be classified as preventable conditions.

Returning to example A, and as applied specifically to the Heart Disease category, a statistical algorithm can be implemented which identifies patients with a higher overall responsibility or contribution to the Heart Disease costs (step 88). Each of the selected patients is given a specialized management plan having a treatment action plan which has been developed for the respective patient (step 90).

From the treatment action plan, system 10 implements an initial benchmark such as a predefined appropriate level of cholesterol at a certain time in the treatment process (step 92). If a targeted employee patient exhibits certain chronic heart conditions (step 94), system 10 can implement a more aggressive treatment action plan (step 96) which can mean additional higher-level testing or a referral to Local Heart Institute, for example.

In either case, method 82 then implements a drug therapy plan (step 98) for the targeted patient, and implements a specialized follow up schedule (step 100) which is communicated to the patient and medical professional. Method 82 then ends (step 102).

Various aspects of the present invention, including the implementation of methods such as method 82 as previously described can be applied not only to employees in a particular onsite health clinic, but also to dependents of the employees or others having an association with the employee. As one skilled in the art will appreciate, various organizations of patients can be created and served using certain aspects of the invention.

Unlike traditional employer health plans, implementation of example method 82 along with other aspects of the present invention brings the element of an onsite medical professional as a health coach to proactively address all ongoing medical issues concerning the employer's population. Certain programs can be purchased by employers in which health assessments are completed annually and data produced to inform individuals of the healthy steps they need to take, however, there is currently no face to face follow up on testing and progress by a medical professional which is scheduled and implemented according to the present invention. The present approach schedules and maintains an onsite clinician that will complete the assessment of the population, work with the targeted individuals, refer when needed to the highest level of testing and care and follow proactively the progress of each case. Ultimately, the present approach will lower costs and create a healthier more productive population.

While one or more embodiments of the present invention have been illustrated in detail, the skilled artisan will appreciate that modifications and adaptations to those embodiments may be made without departing from the scope of the present invention as set forth in the following claims.

What is claimed is:

1. A computer-implemented method for administering medical care for a plurality of onsite clinics, comprising:
aggregating preexisting medical data to generate a plurality of gross health care expenses which are organized according to each of a plurality of health conditions;
prioritizing each of the plurality of gross health care expenses according to cost to generate a health risk assessment;
selecting a pool of associated patients from each of the plurality of gross health care expenses according to a targeted aggregate health care expense;
identifying a first patient of the pool of associated patients to determine an appropriate treatment action plan for the first patient, the treatment action plan consistent with the health risk assessment; and
scheduling onsite consultation, medical testing and benchmarking for the first patient according to the treatment action plan.

2. The method of claim 1, wherein aggregating preexisting medical data to determine a plurality of health care expenses further includes aggregating insurance claims data and/or aggregating self-assessment data.

3. The method of claim 1, further including assigning a certified nurse practitioner (CNP) responsibility for implementing the treatment action plan for the first patient.

4. The method of claim 3, wherein the certified nurse practitioner (CNP) is scheduled to administer care at a first onsite location according to the treatment action plan.

5. The method of claim 1, further including implementing a predetermined contract with a health insurer to provide coverage for the consultation or medical testing, and to provide coverage for follow up medical treatment.

6. The method of claim 5, wherein the health insurer contract excludes or limits coverage for medical testing or medical treatment which is deemed by the health insurer to be experimental, excessively costly or medically unnecessary.

7. The method of claim 5, wherein the predetermined contract includes an incentive for the first patient to initiate the treatment action plan.

8. The method of claim 1, wherein the benchmarking further includes analyzing ongoing medical claims information or medical test data of the first patient.

9. The method of claim 8, wherein the medical claims information is aggregated according to the plurality of patients to generate a report.

10. A computer-implemented method for administering medical care within an onsite clinic, comprising:
combining insurance claims or self assessment data to generate a plurality of aggregate medical costs organized according to each of a plurality of medical conditions;
generating a health risk assessment based on a prioritization of each of the plurality of aggregate medical costs;
targeting a patient pool associated with a first aggregate medical cost of the plurality of aggregate medical costs based on a proportion of selected medical expenses of the patient pool in relation to the first aggregate medical cost;
selecting a first patient of the patient pool to implement a management plan consistent with the health risk assessment; and
implementing a consultation, medical testing, and benchmarking schedule according to the management plan.

11. The method of claim 10, further including assigning a certified nurse practitioner (CNP) to the first patient to provide primary medical care.

12. The method of claim 10, wherein the certified nurse practitioner (CNP) is scheduled to implement a drug therapy regime or initiate a follow up appointment with the first patient.

13. The method of claim 10, wherein the health risk assessment is specific to the onsite clinic, or is aggregated per a plurality of onsite clinics company-wide.

14. An article of manufacture including computer readable program code for administering medical care within an onsite clinic, wherein the code is capable of causing operations to be performed comprising:
combining insurance claims or self assessment data to generate a plurality of aggregate medical costs organized according to each of a plurality of medical conditions;
generating a health risk assessment based on a prioritization of each of the plurality of aggregate medical costs;
targeting a patient pool associated with a first aggregate medical cost of the plurality of aggregate medical costs based on a proportion of selected medical expenses of the patient pool in relation to the first aggregate medical cost;
selecting a first patient of the patient pool to implement a management plan consistent with the health risk assessment; and
implementing a consultation, medical testing, and benchmarking schedule according to the management plan.

15. The article of manufacture of claim 14, further including code capable of assigning a certified nurse practitioner (CNP) to the first patient to provide primary medical care.

16. The method of claim 14, wherein the certified nurse practitioner (CNP) is scheduled to implement a drug therapy regime or initiate a follow up appointment with the first patient.

17. The method of claim 14, wherein the health risk assessment is specific to the onsite clinic, or is aggregated per a plurality of onsite clinics company-wide.

18. A system for administering medical care for an onsite clinic, comprising:
a database module for combining preexisting medical data to generate a plurality of aggregate medical costs organized according to each of a plurality of medical conditions;
a management module connected to the database module, wherein the management module:
generates a health risk assessment based on a prioritization of each of the plurality of aggregate medical costs,
targets a patient pool associated with a first aggregate medical cost of the plurality of aggregate medical costs based on a proportion of selected medical expenses of the patient pool in relation to the first aggregate medical cost, and
selects a first patient of the patient pool to implement a management plan consistent with the health risk assessment; and
a scheduling module connected to the management module for implementing a consultation, medical testing, and benchmarking schedule according to the management plan.

19. The system of claim 18, wherein the management module assigns a certified nurse practitioner (CNP) to the first patient to provide primary medical care.

20. The system of claim 18, wherein the certified nurse practitioner (CNP) is scheduled by the management module to implement a drug therapy regime or initiate a follow up appointment with the first patient.

21. The system of claim 18, wherein the health risk assessment is specific to the onsite clinic, or is aggregated by the management module per a plurality of onsite clinics company-wide.
